(19) United States
(12) Patent Application Publication
(10) Pub. No.: US 2004/0137018 A1
(43) Pub. Date: Jul. 15, 2004

(54) Aspergillus vaccine preparation and methods of making and using thereof

(76) Inventors: James I. Ito, La Verne, CA (US); Joseph M. Lyons, Claremont, CA (US)

Correspondence Address: PERKINS COIE LLP, POST OFFICE BOX 1208, SEATTLE, WA, 98111-1208, US

(21) Appl. No.: 10/639,605

(22) Filed: Aug. 11, 2003

Related U.S. Application Data
(60) Provisional application No. 60/402,445, filed on Aug. 9, 2002.

(51) Int. Cl. A61K 39/00

(52) U.S. Cl. 424/274.1

(57) ABSTRACT

The present invention relates to compositions and methods in preventing and/or treating diseases caused by Aspergillus. In particular, the present invention is directed to Aspergillus vaccine preparations and methods of making and using thereof. Examples of making the Aspergillus vaccine preparation include the step of heating, sonicating or filtrating Aspergillus. Examples of diseases caused by Aspergillus includes invasive aspergillosis. Examples of using the Aspergillus vaccine preparation include the step of vaccinating a subject susceptible to a disease caused by Aspergillus with the Aspergillus vaccine preparation.

### CROSS-REFERENCE TO RELATED APPLICATION

This application claims the benefit of U.S. Provisional Patent Application No. 60/402,445, filed Aug. 9, 2002, which is hereby incorporated by reference in its entirety as fully set forth herein.

### FIELD OF THE INVENTION

The present invention relates to the field of prevention and treatment of diseases caused by Aspergillus. In particular, the present invention relates to the prevention and treatment of aspergillosis using an Aspergillus vaccine preparation.

### BACKGROUND OF THE INVENTION

Invasive aspergillosis, an emerging infection of the immunocompromised host, has devastating consequences. It is especially common among patients receiving corticosteroids for neoplastic disease and during hematopoietic cell transplantation, Baddley, W. et al., Invasive mold infections in allogeneic bone marrow transplant recipients, Clin. Infect. Dis. 32: 1319-24 (2001), and, despite the development and use of new antifungal agents and the implementation of antifungal prophylaxis, the incidence and mortality rates remain high. Lin, J. et al., Aspergillosis case-fatality rate: systematic review of the literature, Clin. Infect. Dis. 32:358-66 (2001).

The first and major lines of defense against inhaled Aspergillus species conidia are the pulmonary macrophage and the neutrophil. Schaffner, A. et al., Selective protection against conidia by mononuclear and against mycelia by polymorphonuclear phagocytes in resistance to Aspergillus: observations on these two lines of defense in vivo and in vitro with human and mouse phagocytes, J. Clin. Invest. 69: 617-31(1982). The immunosuppressive effects of corticosteroids are thought to be due to a suppression of the antimicrobial activity of both macrophages and neutrophils. Schaffner, A., Therapeutic concentrations of glucocorticoids suppress the antimicrobial activity of human macrophages without impairing their responsiveness to gamma interferon, J. Clin. Invest. 76:1755-64 (1985). Roilides, E. et al., Prevention of corticosteroid-induced suppression of human polymorphonuclear leukocyte-induced damage of Aspergillus fumigatus hyphae by granulocyte colony-stimulating factor and gamma interferon, Infect. Immun. 61:4870-7 (1993). Although cytokines such as granulocyte-macrophage colony-stimulating factor (GM-CSF) and interferon (IFN)-γ prevent corticosteroid-induced immunosuppression in vitro (Roilides, supra. See also, Roilides, E. et al., Prevention of corticosteroid-induced suppression of human polymorphonuclear leukocyte-induced damage of Aspergillus fumigatus hyphae by granulocyte colony-stimulating factor and gamma interferon, Infect. Immun. 61:4870-7 (1993)), use of these cytokines has not been shown to restore immunocompetence or to prevent or enhance therapy of invasive aspergillosis. Casadevall, A. & Pirofski, A., Adjunctive immune therapy for fungal infections, Clin. Infect. Dis. 33:1048-56 (2001).

Therefore, there is a need to develop new compositions and methods in preventing or treating diseases caused by Aspergillus.

### SUMMARY OF THE INVENTION

The aspects of the present invention are directed to a composition comprising an Aspergillus vaccine preparation, a method of making an Aspergillus vaccine preparation, and a method of vaccinating a subject susceptible to a disease caused by Aspergillus with the Aspergillus vaccine preparation.

In one embodiment of the invention, an Aspergillus vaccine preparation includes a live Aspergillus, a fraction or a fragment of a live Aspergillus, a heat-treated Aspergillus, a sonicated Aspergillus, and a filtrated Aspergillus. In a preferred embodiment, an Aspergillus vaccine preparation include a sonicated Aspergillus.

In another embodiment of the invention, a method of making an Aspergillus vaccine preparation comprises a step of treating an Aspergillus in a way that will make a subject administered with an Aspergillus vaccine preparation vaccinal or immunoreactive to Aspergillus.

In a preferred embodiment of the invention, a method of making an Aspergillus vaccine preparation comprises a step of heating Aspergillus. In another preferred embodiment of the invention, a method of making the Aspergillus vaccine preparation comprises a step of sonicating Aspergillus. In another preferred embodiment of the invention, a method of making the Aspergillus vaccine preparation comprises a step of filtrating Aspergillus.

In another embodiment of the invention, a method for preventing and/or treating a disease caused by Aspergillus comprises steps of vaccinating a subject with an Aspergillus vaccine preparation. In a preferred embodiment of the invention, a method for preventing and/or treating a disease caused by an Aspergillus comprises steps of administering to a subject with the Aspergillus vaccine preparation.

In another embodiment of the invention, Aspergillus is a species of the Aspergillus genus. In a preferred embodiment of the invention, Aspergillus is Aspergillus fumigatus, Aspergillus flavus, Aspergillus niger, Aspergillus terreus, or Aspergillus midulans.

In another embodiment of the invention, a diseased caused by Aspergillus includes invasive pulmonary aspergillosis, aspergillus tracheobronchitis, invasive aspergillus sinusitis, disseminated aspergillosis, cutaneous aspergillosis, cerebral aspergillosis, and other forms of invasive aspergillosis.

In another embodiment of the invention, an Aspergillus vaccine preparation is administered to a subject through a conjunctival administration, a nasal or intranasal administration, a buccal administration, an oral administration, a rectal administration, a vaginal administration, an epidermal administration, and a parenteral administration that includes a subcutaneous administration, an intramuscular administration, or an intravenous administration. In a preferred embodiment, an Aspergillus vaccine preparation is administered to a subject intranasally or subcutaneously.

In another embodiment of the invention, an Aspergillus vaccine preparation is administered to a subject at any time. In a preferred embodiment of the invention, an Aspergillus vaccine preparation is administered to a subject before the subject becomes immunocompromised and/or immunosupressed. In another preferred embodiment of the invention, an Aspergillus vaccine preparation is administered to a subject prior to the infection of Aspergillus.

In another embodiment of the invention, a subject is a vertebrate. In a preferred embodiment of the invention, the subject is a mammal. In another preferred embodiment, the subject is a mouse. In a more preferred embodiment of the invention, the subject is a human. The subject is a immunocompetent subject or an immunocompromised subject.

Other embodiments are described in the following specification.

### DETAILED DESCRIPTION OF THE INVENTION

The aspects of the present invention are directed to a composition comprising a Aspergillus vaccine preparation, a method of making an Aspergillus vaccine preparation, and a method of vaccinating a subject susceptible to a disease caused by Aspergillus with an Aspergillus vaccine preparation.

In one embodiment of the invention, an Aspergillus vaccine preparation includes a live Aspergillus, a fraction or a fragment of a live Aspergillus, a heat-treated Aspergillus, sonicated Aspergillus, and a filtrated Aspergillus. In a more preferred embodiment, an Aspergillus vaccine preparation comprises a sonicated Aspergillus.

In another embodiment of the invention, a method of making an Aspergillus vaccine preparation comprises a step of treating Aspergillus in a way that will make a subject administered with the preparation vaccinal or immunoreactive to Aspergillus.

In a preferred embodiment of the invention, a method of making an Aspergillus vaccine preparation comprises a step of heating Aspergillus. In another preferred embodiment of the invention, a method of making a Aspergillus vaccine preparation comprises a step of sonicating Aspergillus. In another preferred embodiment of the invention, a method of making a Aspergillus vaccine preparation comprises a step of filtrating Aspergillus.

In another embodiment of the invention, a method for preventing or treating a disease caused by Aspergillus comprises the step of vaccinating a subject with an Aspergillus vaccine preparation. In a preferred embodiment of the invention, a method for preventing or treating a disease caused by Aspergillus comprises the step of administering to a subject with an Aspergillus vaccine preparation.

In another embodiment of the invention, Aspergillus is a species of the Aspergillus genus. In a preferred embodiment of the invention, Aspergillus is Aspergillus fumigatus, Aspergillus flavus, Aspergillus niger, Aspergillus terreus, or Aspergillus midulans.

In another embodiment of the invention, a diseased caused by Aspergillus includes invasive pulmonary aspergillosis, aspergillus tracheobronchitis, invasive aspergillus sinusitis, disseminated aspergillosis, cutaneous aspergillosis, cerebral aspergillosis, and other forms of invasive aspergillosis.

In another embodiment of the invention, a subject is a vertebrate. In a preferred embodiment of the invention, the subject is a mammal. In a more preferred embodiment of the invention, the subject is a human. The subject is a healthy subject or an immunocompromised subject.

In another embodiment of the invention, an Aspergillus vaccine preparation is administered to a subject susceptible to a disease caused by Aspergillus before the subject becomes immunosuppressed or immunocompromised. In another embodiment of the invention, the subject is subject to immunocompromise or immunosuppression after the administering of an Aspergillus vaccine preparation. In a more preferred embodiment, the subject receives corticosteroids for neoplastic disease and/or during hematopoietic cell transplantation after the administering of an Aspergillus vaccine preparation.

In another embodiment of the invention, an Aspergillus vaccine preparation is administered to a subject at any time.

In another embodiment of the invention, an Aspergillus vaccine preparation is administered to a subject with a pharmaceutically acceptable carrier. The Aspergillus vaccine preparation and the pharmaceutically acceptable carrier comprise an Aspergillus vaccine composition.

An Aspergillus vaccine preparation and/or an Aspergillus vaccine composition can be administered to a subject by any administration route known in the art, including without limitation, oral, enteral, buccal, nasal, intranasal, topical, rectal, vaginal, aerosol, transmucosal, epidermal, transdermal, ophthalmic, pulmonary, and/or parenteral administration. For example, the nasal and/or intranasal administration refers to the delivery of an Aspergillus vaccine preparation and/or an Aspergillus vaccine composition across the nasal mucous epithelium and into the peripheral circulation. The conjunctival administration refers to the delivery of an Aspergillus vaccine preparation and/or an Aspergillus vaccine composition across the corneal and conjunctival surface into the eye.

The buccal administration refers to the delivery across the buccal or lingual epithelia into the peripheral circulation. The oral administration refers to the delivery of an Aspergillus vaccine preparation and/or an Aspergillus vaccine composition through the buccal epithelia but predominantly swallowed and absorbed in the stomach and alimentary tract. The rectal administration refers the delivery of an Aspergillus vaccine preparation and/or an Aspergillus vaccine composition via the lower alimentary tract mucosal membranes into the peripheral circulation. The vaginal administration refers to the delivery of an Aspergillus vaccine preparation and/or an Aspergillus vaccine composition through vaginal mucous membrane into the peripheral circulation. A parenteral administration refers to an administration route that typically relates to injection which includes but is not limited to intravenous, intramuscular, intraarterial, intrathecal, intracapsular, intraorbital, intra cardiac, intradermal, intraperitoneal, transtracheal, subcutaneous, subcuticular, intraarticular, subcapsular, subarachnoid, intraspinal, and/or intrasternal injection and/or infusion.

In a more preferred embodiment of the invention, an Aspergillus vaccine preparation and/or an Aspergillus vaccine composition is administered to a subject intranasally or subcutaneously.

The term “pharmaceutically acceptable carrier” as used herein means a pharmaceutically-acceptable material, composition or vehicle, such as a liquid or solid filler, diluent, excipient, solvent or encapsulating material, involved in carrying or transporting an Aspergillus vaccine preparation from one tissue, organ, or portion of the body, to another tissue, organ, or portion of the body. Each carrier must be “pharmaceutically acceptable” in the sense of being compatible with the other ingredients, e.g., an Aspergillus vaccine preparation, of the formulation and suitable for use in contact with the tissue or organ of subjects without excessive toxicity, irritation, allergic response, or other problems or complications, commensurate with a reasonable benefit/risk ratio. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

Typically, an Aspergillus vaccine preparation and/or composition is given to a subject in the form of formulations or preparations suitable for each administration route. The formulations useful in the methods of the present invention include one or more Aspergillus vaccine preparation, one or more pharmaceutically acceptable carriers therefor, and optionally other therapeutic ingredients. The formulations may conveniently be presented in unit dosage form and may be prepared by any methods well known in the art of pharmacy. The amount of active ingredient which can be combined with a carrier material to produce a single dosage form will vary depending upon the subject being treated and the particular mode of administration. The amount of an Aspergillus vaccine preparation which can be combined with a carrier material to produce a pharmaceutically effective dose will generally be that amount of an Aspergillus vaccine preparation which produces a therapeutic effect, which for example allows a subject vaccinal to Aspergillus. Generally, out of one hundred percent, this amount will range from about 1 percent to about ninety-nine percent of the Aspergillus vaccine preparation, preferably from about 5 percent to about 70 percent.

Methods of preparing these formulations or compositions include the step of bringing into association an Aspergillus vaccine preparation with one or more pharmaceutically acceptable carriers and, optionally, one or more accessory ingredients. In general, the formulations are prepared by uniformly and intimately bringing into association an Aspergillus vaccine preparation with liquid carriers, or finely divided solid carriers, or both, and then, if necessary, shaping the product.

Formulations suitable for oral administration may be in the form of capsules, cachets, pills, tablets, lozenges (using a flavored basis, usually sucrose and acacia or tragacanth), powders, granules, or as a solution or a suspension in an aqueous or non- aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of an Aspergillus vaccine preparation as an active ingredient. A compound may also be administered as a bolus, electuary, or paste.

In solid dosage forms for oral administration (e.g., capsules, tablets, pills, dragees, powders, granules and the like), an Aspergillus vaccine preparation is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate, (5) solution retarding agents, such as paraffin, (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered peptide or peptidomimetic moistened with an inert liquid diluent.

Tablets, and other solid dosage forms, such as dragees, capsules, pills and granules, may optionally be scored or prepared with coatings and shells, such as enteric coatings and other coatings well known in the pharmaceutical-formulating art. They may also be formulated so as to provide slow or controlled release of an Aspergillus vaccine preparation therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the Aspergillus vaccine preparation(s) only, or preferentially, in a certain portion of the gastrointestinal tract, optionally, in a delayed manner. Examples of embedding compositions which can be used include polymeric substances and waxes. The Aspergillus vaccine preparation can also be in micro-encapsulated form, if appropriate, with one or more of the above-described excipients.

Liquid dosage forms for oral administration include pharmaceutically acceptable emulsions, microemulsions, solutions, suspensions, syrups and elixirs. In addition to the Aspergillus vaccine preparation, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcoho, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, coloring, perfuming and preservative agents.

Suspensions, in addition to an Aspergillus vaccine preparation, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations for rectal or vaginal administration may be presented as a suppository, which may be prepared by mixing one or more Aspergillus vaccine preparation with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active agent. Formulations which are suitable for vaginal administration also include pessaries, tampons, creams, gels, pastes, foams or spray formulations containing such carriers as are known in the art to be appropriate.

Formulations for the topical or transdermal or epidermal administration of an Aspergillus vaccine composition include powders, sprays, ointments, pastes, creams, lotions, gels, solutions, patches and inhalants. The active component may be mixed under sterile conditions with a pharmaceutically acceptable carrier, and with any preservatives, buffers, or propellants which may be required. The ointments, pastes, creams and gels may contain, in addition to an Aspergillus vaccine composition, excipients, such as animal and vegetable fats, oils, waxes, paraffins, starch, tragacanth, cellulose derivatives, polyethylene glycols, silicones, bentonites, silicic acid, talc and zinc oxide, or mixtures thereof. Powders and sprays can contain, in addition to the Aspergillus vaccine composition, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Aspergillus vaccine compositions can be alternatively administered by aerosol. This is accomplished by preparing an aqueous aerosol, liposomal preparation or solid particles containing Aspergillus vaccine preparation. A nonaqueous (e. g., fluorocarbon propellant) suspension could be used. Sonic nebulizers can also be used. An aqueous aerosol is made by formulating an aqueous solution or suspension of the agent together with conventional pharmaceutically acceptable carriers and stabilizers. The carriers and stabilizers vary with the requirements of the particular compound, but typically include nonionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions.

Formulations suitable for parenteral administration comprise an Aspergillus vaccine preparation in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacterostats, solutes which render the formulation isotonic with the blood of the intended recipient or suspending or thickening agents.

Examples of suitable aqueous and nonaqueous carriers which may be employed in the formulations suitable for parenteral administration include water, ethanol, polyols (e.g., such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

Formulations suitable for parenteral administration may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the vaccinal effect of an Aspergillus vaccine preparation, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered formulation is accomplished by dissolving or suspending the Aspergillus vaccine composition in an oil vehicle.

Injectable depot forms are made by forming microencapsule matrices of an Aspergillus vaccine preparation or in biodegradable polymers such as polylactide-polyglycolide. Depending on the ratio of the Aspergillus vaccine preparation to polymer, and the nature of the particular polymer employed, the rate of drug release can be controlled. Examples of other biodegradable polymers include poly (orthoesters) and poly (anhydrides). Depot injectable formulations are also prepared by entrapping an Aspergillus vaccine preparation or composition in liposomes or microemulsions which are compatible with body tissue.

In a preferred embodiment of the invention, an Aspergillus vaccine composition is delivered to a subject in a therapeutically effective dose. The term “pharmaceutically effective dose” as used herein refers to the amount of the Aspergillus vaccine preparation and/or an Aspergillus vaccine composition, which is effective for producing a desired vaccinal effect. As is known in the art of pharmacology, the precise amount of the pharmaceutically effective dose of an Aspergillus vaccine preparation that will yield the most effective results in terms of efficacy of treatment in a given subject will depend upon, for example, the activity, the particular nature, pharmacokinetics, pharmacodynamics, and bioavailability of a particular Aspergillus vaccine preparation, physiological condition of the subject (including race, age, sex, weight, diet, disease type and stage, general physical condition, responsiveness to a given dosage and type of medication), the nature of pharmaceutically acceptable carriers in a formulation, the route and frequency of administration being used, to name a few. However, the above guidelines can be used as the basis for fine-tuning the treatment, e.g., determining the optimum dose of administration, which will require no more than routine experimentation consisting of monitoring the subject and adjusting the dosage. Remington: The Science and Practice of Pharmacy (Gennaro ed. 20^{th} edition, Williams & Wilkins PA, USA) (2000).

Having generally described the present invention, the same will be better understood by reference to certain specific examples, which are set forth herein for the purpose of illustration.

### EXAMPLES

Invasive pulmonary aspergillosis is an emerging devastating infection in the immunocompromised host that is treated with corticosteroids for neoplastic disease or for organ transplantation. One approach to overcoming corticosteroid-induced immunosuppression is to vaccinate against Aspergillus species prior to corticosteroid administration with the hope that a sensitized immune system might be able to overcome this immunosuppression, perhaps by responding with enough cytokines to reverse the defect in antifungal activity of these granulocytes.

The present study used a model of invasive pulmonary aspergillosis in corticosteroid-treated mice to test the efficacy of prior infection and two Aspergillus fumigatus vaccine preparations administered via two different routes in protecting against subsequent lethal Aspergillus species infection.

By use of a model of invasive pulmonary aspergillosis in corticosteroid-treated CF-1 mice, prior infection and 2 Aspergillus fumigatus vaccine preparations (sonicate and filtrate) administered intranasally and subcutaneously were tested for efficacy in protecting against subsequent lethal A. fumigatus infection. The mortality rates were as follows: control subjects, 100%; prior infection, 12.5%; sonicate administered intranasally, 29%; sonicate given subcutaneously, 0%; filtrate given intranasally, 75%; and filtrate given subcutaneously, 50%. Prior infection and A. fumigatus sonicate vaccine administered by 2 routes protected corticosteroid-treated animals against subsequent lethal invasive pulmonary aspergillosis. The sonicate vaccine was more protective, but the subcutaneous route was more effective.

### Materials and Methods

Mice. Female CF-1 mice were purchased from Charles River Breeding Laboratories and were used at ages 7-8 weeks.

A. fumigatus. A strain of A. fumigatus isolated from a patient with invasive pulmonary aspergillosis at the City of Hope National Medical Center (Duarte, Calif.) was used for vaccine preparations and infection. Conidia were collected in sterile 0.9% saline containing 0.1% Tween 80 from 5-7-day cultures on potato dextrose agar plates grown at 37° C. Clumps of conidia were dispersed with 3-mm glass beads, and the suspension was washed twice and suspended to the desired concentration with 0.9% saline containing 0.01% Tween 80. This procedure gave mycelia-free suspensions of conidia with >95% single conidia. Conidia were enumerated with a hemocytometer, and viability was assessed by agar plating. The washed hyphal mass was sonicated 3 times, for 2 minutes, on ice, with a Bronwill Biosonik III Sonicator at 30% intensity level using a 4 mm flat bottom sonicator tip. Prior to and between cycles the hyphal material was frozen at −80° C. and quickly thawed at 37° C. and placed on ice; and following sonication the hyphal sonicate or the sonicated Aspergillus was aliquoted and frozen at −80° C.

Vaccines and vaccination. Mice were vaccinated twice, 2 weeks apart, 3 weeks before infectious challenge, either intranasally with 30 μL or subcutaneously at 2 sites with 100 μL of the following vaccine preparations: 10⁶ viable conidia administered intranasally only (prior infection), 7-day liquid-culture-grown hyphal mass disrupted by 3 freeze-thaw and sonication cycles (sonicate or sonicated Aspergillus), and filter sterilized and 200-times concentrated 14-day liquid culture supernatant (filtrate). Intranasal administration was done under light ketamine/xylazine anesthesia.

Treatments. Cortisone acetate was administered subcutaneously in 2.5-mg doses for 6 consecutive days prior to challenge (Schaffner, A. et al., Selective protection against conidia bymononuclear and against mycelia by polymorphonuclear phagocytes in resistance to Aspergillus: observations on these two lines of defense in vivo and in vitro with human and mouse phagocytes, J.

Clin. Invest. 69: 617-31(1982)), commencing 2 weeks after the second immunization. To reduce risk of bacterial infection, mice were administered 200 μg of levofloxacin subcutaneously 1 h prior to infection.

Infection. Under light ketamine/xylazine anesthesia, mice were intranasally inoculated with 30 mL of conidial suspension containing 10⁶ viable conidia while being held in the vertical position and were placed on their backs during recovery from anesthesia. After challenge, all animals fully recovered within 1-2 h and were normal in appearance until signs of disease became apparent 24-30 h after inoculation.

Assessment of infection. After inoculation, mice were observed on a regular basis during the day, and the time of death was recorded. Deaths that occurred at night were assigned a time of death coinciding with the first morning observation. The lungs of animals were cultured to ensure that the cause of death was invasive aspergillosis. Data were analyzed by use of Fisher's exact test.

### Results

Animals that were previously infected or were vaccinated with sonicate vaccine were significantly protected against fatal invasive pulmonary aspergillosis (See, Table I). Although there was a trend toward protection in animals vaccinated with filtrate vaccine, significant protection was not demonstrated. The sonicate vaccine was more effective than the filtrate vaccine (P=0.01), and the subcutaneous route of vaccination appeared to be more effective than the intranasal route, but the difference did not reach significance (P=0.16)

### Discussion

Prior infection and two A. fumigatus vaccine preparations conferred protection against lethal challenge and overcame the immunosuppressive effects of corticosteroids. The immunologic mechanism by which this occurs is unknown. One possibility is that the sensitized animal, after rechallenge with A. fumigatus antigen(s), is capable of specifically responding with larger quantities of cytokines (e.g., IFN-γ and GM-CSF) that might reverse the antimicrobial defect in macrophages and neutrophils induced by corticosteroids. Roilides, E. et al., Prevention of corticosteroid-induced suppression of human polymorphonuclear leukocyte-induced damage of Aspergillus fumigatus hyphae by granulocyte colony-stimulating factor and gamma interferon, Infect. Immun. 61:4870-7 (1993). Roilides, E., et al., Granulocyte-macrophage colony-stimulating factor and interferon-gamma prevent dexamethasoneinduced immunosuppression of antifungal monocyte activity against Aspergillus fumigatus hyphae, J. Med. Vet. Mycol. 34:63-9 (1996).

Recently, Cenci et al. demonstrated that vaccination with an A. fumigatus culture filtrate was capable of inducing protection in a neutropenic murine model of invasive pulmonary aspergillosis. Cenci, E. et al., T cell vaccination in mice with Pulmonary aspergillosis, J. Immunol. 165:381-8 (2000). They showed that protection could be conferred by the adoptive transfer of antigen-specific CD4 T cells producing IFN-γ and interleukin-2. More significantly, they observed local recruitment of lymphocytes and macrophages, despite a profound leukopenia.

Thus, in both models, the mechanism may be similar. During subsequent challenge, antigen-specific CD4 T cells expressed increased the amounts of cytokines. Both recruited more cells to the area of infection and enhanced microbicidal activity of granulocytes and, in one model, overcame a systemic neutropenia and, in the other, overcame the immunosuppressive effect of corticosteroids.

It is of interest that the subcutaneous route of immunization appears to be superior to the intranasal route, the natural portal of entry for this pathogen. But there is precedence for this phenomenon in the field of vaccination against Chlamydia trachomatis genital tract infection. Igietseme, U. et al., Route of infection that induces a high intensity of gamma interferon-secreting T cells in the genital tract produces optimal protection against Chlamydia trachomatis infection in mice, Infect. Immun. 66:4030-5 (1998).

In conclusion, vaccination with an A. fumigatus antigen preparation and prior infection can confer protection against subsequent lethal invasive pulmonary aspergillosis in the setting of corticosteroid immunosuppression. In this study, the sonicate vaccine was more protective than the filtrate vaccine, and the subcutaneous route appeared to be more effective than the intranasal route.

TABLE 1
Mortality from invasive pulmonary aspergillosis after
vaccination or infection.

| | No. | Cumulative | | | | | | | | |
|---|---|---|---|---|---|---|---|---|---|---|
| | of | deaths after infections, days | | | | | | | Mortality, | |
| Group | mice | 1 | 2 | 3 | 4 | 5 | 6 | 7 | % | p^{a} |
| Control | 8 | 0 | 3 | 8 | 8 | 8 | 8 | 8 | 100 | — |
| Prior Infection | 8 | 0 | 1 | 1 | 1 | 1 | 1 | 1 | 12.5 | .001 |
| Sonicate | | 0 | 2 | 2 | 2 | 2 | 2 | 2 | 29 | .01 |
| Intranasal | 7 | | | | | | | | | |
| Subcutaneous | 8 | 0 | 0 | 0 | 0 | 0 | 0 | 0 | 0 | .0002 |
| Filtrate | | | | | | | | | | |
| Intranasal | 8 | 0 | 3 | 5 | 6 | 6 | 6 | 6 | 75 | .46 |
| Subcutaneous | 8 | 0 | 2 | 2 | 2 | 2 | 2 | 4 | 50 | .076 |

*Fisher's exact test (vs. control).

Papers and patents listed in the disclosure are expressly incorporated by reference in their entirety. It is to be understood that the description, specific examples, tables and figures, while indicating preferred embodiments, are given by way of illustration and exemplification and are not intended to limit the scope of the present invention. Various changes and modifications within the present invention will become apparent to the skilled artisan from the disclosure contained herein. Therefore, the spirit and scope of the appended claims should not be limited to the description of the preferred versions contained herein.

I/We claim:

1. An Aspergillus vaccine preparation, for preventing a disease caused by Aspergillus, comprising a sonicated Aspergillus.

2. The Aspergillus vaccine preparation of claim 1 wherein the disease is selected from the group consisting of invasive pulmonary aspergillosis, aspergillus tracheobronchitis, invasive aspergillus sinusitis, disseminated aspergillosis, cutaneous aspergillosis, and cerebral aspergillosis.

3. A method of preventing a disease caused by Aspergillus in a subject comprising the step of administering to the subject with an Aspergillus vaccine preparation.

4. The method of claim 3 wherein the Aspergillus vaccine preparation is a sonicated Aspergillus.

5. The method of claim 3 wherein the disease caused by Aspergillus is selected from the group consisting of invasive pulmonary aspergillosis, aspergillus tracheobronchitis, invasive aspergillus sinusitis, disseminated aspergillosis, cutaneous aspergillosis, and cerebral aspergillosis.

6. The method of claim 3 wherein the Aspergillus vaccine preparation is administered to the subject through an administration route selected from the group consisting of intranasal, nasal, oral, enteral, buccal, topical, rectal, vaginal, aerosol, transmucosal, epidermal, transdermal, ophthalmic, pulmonary, and/or parenteral administration.

7. The method of claim 6 wherein the parenteral administration is selected from the group consisting of subcutaneous, intravenous, intramuscular, intraarterial, intrathecal, intracapsular, intraorbital, intra cardiac, intradermal, intraperitoneal, transtracheal, subcuticular, intraarticular, subcapsular, subarachnoid, intraspinal, and/or intrasternal injection and/or infusion.

8. The method of claim 3 wherein the Aspergillus vaccine preparation is administered to the subject intranasally or subcutaneously.

9. The method of claim 3 wherein the Aspergillus vaccine preparation is administered prior to the immunocompromise or immunosuppression of the subject.

10. A method of preventing invasive pulmonary Aspergillosis in a subject comprising a step of administering to the subject with a sonicated Aspergillus vaccine preparation.

11. The method of claim 10 wherein the sonicated Aspergillus vaccine preparation is administered intranasally.

12. The method of claim 10 wherein the sonicated Aspergillus vaccine preparation is administered subcutaneously.

13. The method of claim 10 wherein the sonicated Aspergillus vaccine preparation is administered prior to the immunocompromise of the subject.

14. The method of claim 13 wherein the immunocompromise is effected by corticosteroid.

15. The method of claim 13 wherein the immunocompromise is effected by hematopoietic cell transplantation.

16. The method of claim 10 wherein the sonicated Aspergillus vaccine preparation is made by a sonicator.
